(19) United States
(12) Patent Application Publication
(10) Pub. No.: US 2003/0199858 A1
(43) Pub. Date: Oct. 23, 2003

(54) Multifocal refractive surgery optimized to pupil dimensions and visual acuity requirements

(76) Inventor: Lee Paul Schelonka, Lakewood, CO (US)

Correspondence Address: LEE P. SCHELONKA, 3083 SOUTH OAK WAY, LAKEWOOD, CO, 80227, US

(21) Appl. No.: 10/126,379

(22) Filed: Apr. 18, 2002

(51) Int. Cl. A61B 18/18

(52) U.S. Cl. 606/5; 606/10; 128/898

(57) ABSTRACT

Multifocal corneal refractive surgery for the correction of presbyopia is optimized, based on patient pupil measurements and acuity requirements. Measurements are made of the patient's pupil dimensions in bright and dim light, with near and distant focusing. A series of mathematical models of the wavefront transmitted through the eye/multifocal optic system is constructed, and the modulation transfer functions are calculated, for a series of optical zone dimensions and decentrations. The maximum resolvable spatial frequency and the expected visual acuity are calculated as functions of the zone dimensions and decentration. The patient's near and distant visual acuity requirements are compared to the expected visual acuity, and the optimized zone dimensions and decentration meeting the acuity requirements are determined. A required postoperative multifocal corneal profile is calculated. A computer-controlled laser, mechanical, thermal, or conductive device reshapes the cornea or a corneal implant. Nomograms are disclosed for centered, circular multifocal refractive surgery.

### BACKGROUND OF THE INVENTION

This invention relates to the optics of the eye, the cornea, and refractive surgery. It specifies an improved method to create a multifocal cornea, with optical zone dimensions and decentration optimized, based on measurements of the patient's pupils and the patient visual acuity requirements. The refractive surgery corrects myopia, hyperopia, regular astigmatism and higher order aberrations, in addition to presbyopia.

The visual acuity required by a patient depends on the visual tasks. For distant tasks such as driving, many patients require 20/20 acuity. The Snellen units of 20/20 correspond to a minimum angle of resolution of about one arc minute, a maximum resolvable spatial frequency of about 30 cycles per degree, and a decimal acuity of 1.0. However, many near visual tasks require a lower level of acuity. For example, reading seven point lowercase printed matter at a standard distance of 14 inches (0.36 meters) requires only about 20 cycles per degree of angular resolution. This is equivalent to about 0.67 decimal acuity, or 20/30 distant acuity, and is termed Jaeger 2 or J2 near acuity.

The visual acuity at near depends on accommodation, which is the eye's ability to change focus, and on the depth of focus of the eye. With age, the eye's ability to accommodate declines, resulting in presbyopia. Correcting for near vision requires additional optical power, increasing with increasing age, as in Table 1 (Ref Strauss). Between the ages of 40 and 60, an additional 1.0 Diopter of add power is required for each decade of life. The depth of focus of the eye, also termed pseudoaccommodation or apparent accommodation, is inversely proportional to the required visual acuity and depends also on the pupil diameter and the optics of the eye, including uncorrected refractive error and aberrations (Refs Green (1980) and Fukuyama). Conventionally, presbyopia has been treated with bifocal, trifocal or multifocal glasses or contact lenses.

As patients prefer to decrease their dependence on glasses and contact lenses, corneal refractive surgery has been developed. Corneal refractive surgery often consists of using a laser, scalpel or other modality to sculpt, remove or distort corneal tissue, in order to change its shape. Alternatively, intracorneal implants are placed, changing the corneal power. Laser corneal sculpting may be performed in conjunction with removal of the corneal epithelium, as in photorefractive keratectomy, or after creation of a partial thickness corneal flap, as in laser in-situ keratomileusis and laser subepithelial keratectomy. The corrections of myopia, hyperopia, astigmatism and aberrations with corneal refractive surgery are well known. Recently, these techniques have been modified to produce a multifocal cornea, correcting presbyopia.

In multifocal refractive surgery, at least one region or zone of the cornea predominantly corrects distant vision, and at least one “add zone” has additional optical power to predominantly correct near vision. Within each zone, the optical power can be constant or variable. The radial or spatial profile of optical power may have abrupt transitions from near to distant correction, as in bifocal or trifocal refractive surgery, or can vary smoothly, as in progressive or aspheric multifocal refractive surgery. Although multifocal refractive surgery can improve the reading acuity (Refs Anschutz, Bauerberg, Lindstrom), certain patients do not obtain satisfactory near and distant acuity under all lighting conditions, while others are disturbed by glare and halo effects (Refs Anschutz, Steinert, Lindstrom).

Multifocal refractive surgery produces a focused image, surrounded by a blurred region, potentially causing glare and halos. FIG. 1 shows a multifocal optic with a pupil 10, a zone 11 corrected for near, and a zone 12 corrected for distance, imaging a distant object 13. The zone corrected for distance forms an image 14 of the object, while the zone corrected for near forms a blurred image or halo 15. The power (in Watts) of light in the halo is proportional to the fraction of the pupil area covered by the out-of-focus optical zones. For example, during night driving, the pupil is at its maximum and the focus is distant. Minimizing the size of the add zones and using aspheric transitions of optical power between zones may reduce the prominence of halos. Also, in order to further reduce glare and halos, the dimension of the outermost corrected optical zone should exceed the pupil diameter in dim light with distant focusing (U.S. Pat. No. 6,190,375).

The visual acuity obtained with multifocal ophthalmic optics depends strongly on the sizes of the pupil and the optical zones, and the decentration of the optics from the pupil (Ref. Woods). By way of example, FIG. 2 shows the expected near and distant visual acuity versus the pupil dimensions, for a circular bifocal corneal refractive surgery algorithm, with a 1.7 mm diameter central optical zone corrected for near vision. The expected distant acuity exceeds 1.0 (Snellen 20/20) only when the pupil exceeds a certain size. Similarly, the expected near acuity exceeds 0.67 (Jaeger 2) only when the pupil is smaller than a certain size. Patients with pupils outside these dimensions could suffer unacceptable reductions in their visual acuity if refractive surgery using these dimensions were performed. Measurements of populations of patients have shown that, on average, pupil diameters decrease with increasing light, increasing age, and near focusing. The pupil diameter in bright light decreases about 0.3 mm every 10 years (Ref Koch). The method of this patent sizes and locates the zones based on the pupil dimensions and acuity requirements, and corrects presbyopia as the pupil size declines with age.

Certain aberrations, including spherical aberration and uncorrected myopic astigmatism, may increase the depth of focus of an ophthalmic optical system (Ref Fukuyama). Other aberrations degrade the defocused modulation transfer function, and reduce the expected visual acuity. This patent discloses a method to incorporate the aberrations of the eye in optimizing the multifocal optic zone dimensions.

Perfect centration of corneal refractive surgery over the pupil or visual axis is not possible. Laser in-situ keratomileusis (Lasik) has an average decentration of 0.6-0.7 mm (Ref Lee). Intracorneal implants may also decenter. Intentional decentration of refractive surgery may improve the near acuity (Refs Anschutz and Bauerberg, and U.S. Pat. Nos. 5,533,997, 5,803,923, 5,928,129, and 6,302,877). However, decentration induces astigmatism and may reduce the distant visual acuity (Refs Anschutz and Woods). Using the method of this patent, the decentration is optimized.

A number of techniques of multifocal corneal refractive surgery are known. Multifocal laser corneal sculpting may be performed with the use of masks, diaphragms, scanning laser spots or offset imaging. The geometry of the optical zones may include a sickle shape (U.S. Pat. No. 5,314,422), sector shape (U.S. Pat. No. 6,190,374), kidney bean shape (U.S. Pat. No. 5,803,923), circular, ovoid or annular shape (U.S. Pat. No. 6,059,775), and undisclosed shapes (U.S. Pat. Nos. 5,395,356 and 6,258,082). However, each of these methods fails to address the important role of the pupil diameter in determining the dimensions of the optical zones. Early clinical trials of multifocal corneal refractive surgery have noted that some patients lose visual acuity at distance (Anschutz). This may occur if the sizes, shapes and locations of the optical zones are not ideally proportioned to the patient's pupil dimensions.

Recently, multifocal refractive surgery algorithms have taken into account patient pupil dimensions. In one method, a central optical zone of the cornea is surgically corrected predominantly for near vision, a peripheral optical zone is corrected predominantly for distance vision, and an aspheric blend zone lies between the two zones. The entire optical ablation and the central optical zone are scaled to a dimension of the pupil (U.S. Pat. No. 6,280,435). However, I have discovered that, if this geometry is used, maximum diameter of the central optical zone should be sized, based on the patient's distant visual acuity requirement and the pupil dimension in bright light with distant focusing. The minimum dimension of the central optical zone should be sized, based on the patient's near visual acuity requirement and the pupil dimension in dim light with near focusing. The overall dimension of the treatment should be greater than the dimension of the pupil in dim light with distant focusing. Scaling the optical zones based on a single measurement of the pupil may not provide adequate near and distant visual acuity. This patent discloses a method and nomograms for sizing and locating the zones, regardless of their number or arrangement, to provide adequate near and distant visual acuity.

In U.S. Pat. Nos. 5,533,997, 5,928,129, and 6,302,877 a decentered, inner circular zone and a peripheral zone are left without correction, and an annular zone surrounding the central zone is corrected for near vision. The zones are sized and located, based on the observation that the central 3 mm of many patients' pupils could be occluded, with preservation of good distant acuity. However, these methods may not provide adequate near and distant acuity for patients with small pupils. I have discovered that it is necessary to size and locate the zones based on the near and distant visual acuity requirements, and measurements of the pupil dimensions in bright and dim light with near and distant focusing.

Multifocal intracorneal implants have been disclosed (U.S. Pat. Nos. 5,628,794 and 6,090,141). The implants can consist of small diameter optics correcting only presbyopia, or larger optics, correcting both near and distant vision. If the optics correct only presbyopia, the power in the zones corrected primarily for distance is zero, and the power in the zones corrected for near is the patient's current add power.

Intracorneal implants may be manufactured prior to implantation (U.S. Pat. Nos. 5,628,794 and 6,090,141), or they may be sculpted by a laser or other means after being placed on or in the cornea (U.S. Pat. Nos. 6,063,073 and 6,197,019). Clinical trials have shown improvement in reading vision with intracorneal implants. However, some patients lose uncorrected distant visual acuity with these lenses (Lindstrom). Optimizing the zone dimensions, as disclosed in this patent, may provide acceptable near and distant acuity for a larger proportion of patients.

Certain methods have been disclosed to analyze the performance of ophthalmic optics. The point spread function can be calculated, and convoluted with images to simulate the blur of a multifocal lens and an eye (U.S. Pat. No. 5,677,750). However, this method has not been applied to the problem of optimizing the zone sizes and location, based on measurements of patient pupil dimensions and patient visual acuity requirements. Ray tracing can be used to calculate the surface shape of multifocal optics and correct the aberrations of the eye (U.S. Pat. No. 6,215,096), but this technique ignores diffraction and cannot predict the visual acuity obtained by the eye and the multifocal optic.

A neural network model has been developed to optimize certain designs of multifocal contact lenses for populations of patients (U.S. Pat. No. 5,724,258). However, this method is limited by the data input to it. Unless the inputs to the network explore the full range of multifocal corneal optics, including two, three, four and more optical zones, over the full range of zone dimensions, powers, asphericities and patient pupil dimensions, the optimization will be limited to a restricted set of values. Finally, since contact lenses decenter and move on the surface of the eye, it may be invalid to extrapolate the results of the network to multifocal refractive surgery.

The performance of an optical system can be described by its modulation transfer function, which is the contrast transmitted by the system as a function of the image spatial frequency (Refs Born, Gaskill, Holladay, Woods). FIG. 3 shows an example modulation transfer function 31, a schematic of a contrast threshold function 32, and a maximum resolvable spatial frequency 33. Typically, the transfer function declines with increasing spatial frequency, although there may be fluctuations. In contrast, the average retinal contrast threshold increases with increasing spatial frequency (Ref Green (1978)). The spatial frequency at which the modulation transfer reduces the contrast to the threshold is the maximum resolvable spatial frequency. The expected visual acuity of the eye/multifocal optic system is proportional to the maximum resolvable spatial frequency, with about 30 cycles per degree corresponding to a decimal acuity of 1.0 or 20/20 Snellen acuity (Lang). This invention uses calculations of modulation transfer functions and the expected visual acuity to optimize the size and location of the optical zones in multifocal refractive surgery.

### BRIEF SUMMARY OF THE INVENTION

This invention consists of a method to perform multifocal refractive surgery on the cornea of an eye, to correct myopia, hyperopia, regular and irregular astigmatism, and presbyopia, using optimized optical zones. The patient's near and distant acuity requirements are determined, and the patient's pupils are measured in bright and dim light with near and distant focusing. The refractive error and, preferably, the aberrations of the patient's eyes, the depth of focus of the eyes, and the patient's contrast threshold are also measured and recorded. Candidate geometries are selected, including circular and noncircular shapes, having abrupt or smooth transitions of optical power, centered or decentered from the pupil.

Using the measurements, together with focusing for near or distance, and a series of optical zone dimensions and decentrations, a series of mathematical models of the wavefront transmitted through the eye/multifocal optical system is created. From the wavefronts, a series of modulation transfer functions are calculated. The maximum spatial frequency, at which the modulation transfer function reduces the contrast to the retinal contrast threshold, is calculated. The expected visual acuity is calculated at near and distance, as a function of the optical zone dimensions. It is proportional to the maximum spatial frequency, with about 30 cycles per degree corresponding to a decimal acuity of 1.0. Tables or graphs of the expected near and distant visual acuity versus the zone dimensions and decentration are created. The patient's visual acuity requirements are compared to the expected visual acuity as a function of the zone dimensions and decentration. The minimum and maximum zone dimensions are the dimensions at which the expected acuity falls below the required acuity. This gives the acceptable range or tolerance of zone dimensions. Within the tolerances, the zone sizes are optimized to maximize near or distant visual acuity, minimize glare and halo side effects, maximize the reliability of the procedure, or provide blended multifocal aspheric transition zones or trifocal zones. Similarly, the minimum and maximum decentrations are calculated.

With the zone sizes and decentration determined, an algorithm or table is created, giving the required postoperative corneal profile. This is used to guide a computer-controlled laser, thermal or other device to reshape the cornea or to design or reshape a corneal implant. The zone dimensions and add powers are adjusted to provide continued correction of presbyopia as the pupil declines with age and the add power requirement increases with age. Selecting the optimum zone dimensions, decentration, power profiles and zone arrangement solves the problems noted in previous clinical trials of multifocal refractive surgery. Specifically, the method of this patent improves the near and distant visual acuity for each patient, it reduces the side effects of glare and halos, and it decreases the likelihood that patients will lose distant visual acuity following surgery. It also identifies patients who would be poor candidates for multifocal refractive surgery, either due to unrealistic expectations or due to extremely small pupil dimensions.

Example nomograms are disclosed, giving the allowable zone dimensions and their tolerances for centered, circular multifocal refractive surgery, for a range of patient pupil diameters and visual acuity requirements. The example nomograms for two- and three-zone refractive surgery are based on individual patient measurements, while the nomograms for four and five zones are based on the range of pupil measurements for the entire population. Decentering the zones may allow the use of a single set of zone sizes for two- and three-zone refractive surgery for all patients.

### BRIEF DESCRIPTION OF THE SEVERAL VIEWS OF THE DRAWINGS

FIG. 1 is a schematic of multifocal refractive surgery, forming an image and a halo, with a distant object.

FIG. 2A is a graph of the expected distant visual acuity as a function of the pupil diameter, for an example circular, bifocal refractive surgery.

FIG. 2B is a graph of the expected near visual acuity as a function of the pupil diameter, for an example circular, bifocal refractive surgery.

FIG. 3 is a graph of an example multifocal modulation transfer function, the average human retinal contrast threshold, and a maximum resolvable spatial frequency.

FIG. 4A is a plan view of a two-zone multifocal refractive surgery algorithm, with the central zone corrected for near vision.

FIG. 4B is a graph of example multifocal power profiles, for two-zone, center-near refractive surgery.

FIG. 5A is a graph of the expected distant visual acuity as a function of the central zone diameter, for an example two-zone, center-near bifocal refractive surgery.

FIG. 5B is a graph of the expected near visual acuity as a function of the central zone diameter, for an example two-zone, center-near bifocal refractive surgery.

FIG. 6A is a plan view of a two-zone multifocal refractive surgery algorithm, with the central zone corrected for distant vision.

FIG. 6B is a graph of an example bifocal power profile, for two-zone, center-distant refractive surgery.

FIG. 7A is a plan view of a three-zone multifocal refractive surgery algorithm, with the central zone corrected for distant vision.

FIG. 7B is a graph of example multifocal power profiles, for three-zone, center-distant refractive surgery.

FIG. 8A is a plan view of a four-zone multifocal refractive surgery algorithm, with the central zone corrected for near vision.

FIG. 8B is a graph of example multifocal power profiles, for four-zone, center-near refractive surgery.

FIG. 9A is a plan view of a five-zone multifocal refractive surgery algorithm, with the central zone corrected for distant vision.

FIG. 9B is a graph of example multifocal power profiles, for five-zone, center-distant refractive surgery.

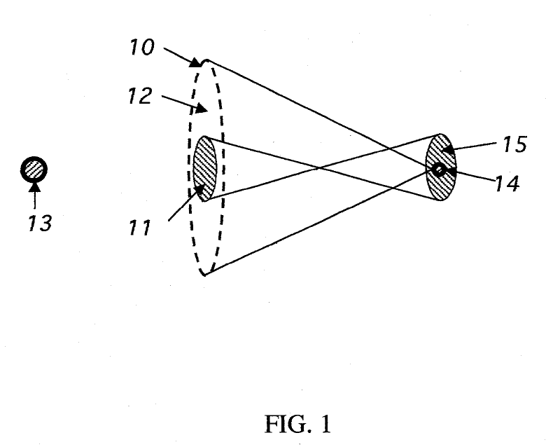

### DETAILED DESCRIPTION OF THE INVENTION

Discussions are held with the patient regarding near and distant acuity requirements under bright and dim lighting conditions. Sample text of various sizes is provided, and the required near acuity may be based on the minimum required type size. The patient should consider the tradeoff of some loss of distant acuity, to improve the near acuity. In general, the less stringent the patient's near acuity requirements and the larger the patient's pupils, the better the distant acuity will be. Following calculation of the optimized zone dimensions and prediction of the near and distant acuity, further discussions may be necessary. The patient may have to revise the near acuity requirement in order to maintain a certain distant acuity, or may be advised that he or she may not be a good candidate for the procedure.

The right and left pupil dimensions are measured in bright and dim lighting, with near and distant focusing. The accuracy of the pupil measurements should be about 0.1 mm. Commercially available pupillometers have this resolution. The lighting and focusing conditions should be standardized (Ref Koch). If the pupil is oval or irregular, its smallest dimension is used in the optimization algorithms. However, the overall dimension of the outermost corrected optical zone is chosen to exceed the largest pupil dimension in dim light with distant focusing.

The refractive error of each eye is measured. Preferably, the aberrations of the right and left eye are measured, and the calculated wavefront of the eye is input directly into the computer, which performs the optimization (Ref Liang). A number of devices are commercially available to measure the aberrations of the eye. Alternatively, the aberrations of the patient's eyes are assumed to be negligible.

One or more candidate optical zone geometries are chosen for investigation, such as circular, sectoral, ovoid, and the like. Two, three or more optical zones may be used. At least one zone has a nominal near correction power, including astigmatism. At least one zone has a nominal distant correction power. The add power depends on the patient's age. In order to continue to correct presbyopia as it worsens with age, additional add power is selected, if tolerated by the patient using trial lenses preoperatively. About 1.0 Diopters of extra add power is required by patients aged 40-50, for each additional decade of desired presbyopia correction, up to a maximum of about 2.75 to 3.0 Diopters. The arrangement of the zones may have the central zone corrected for distance, near, or intermediate focus, the peripheral zone corrected for near, distance, or intermediate focus, and any other zones corrected as desired. Often, correcting the peripheral zone for distance reduces glare and halos.

Preferably, the depth of focus of the right and left eye is measured. The visual acuity of each patient is measured as a function of the distance to a target, or as a function of the optical power of a defocusing lens with an object at a constant distance. By instilling cycloplegic drops onto the eyes, accommodation can be temporarily blunted and the depth of focus of the eye can be determined. It may be necessary to introduce artificial pupil apertures in front of the eyes for these measurements, to determine whether the depth of focus depends strongly on the pupil dimensions. Alternatively, the average depth of focus of the eye, as known in the literature (Refs Green (1980), Fukuyama), is used. Since the depth of focus is inversely proportional to the required acuity and since many patients have lower acuity requirements at near than at distance, the zones corrected for near may have a larger depth of focus than those corrected for distance.

One or more candidate multifocal optical power profiles as a function of the location in the cornea are chosen for investigation, such as centered or decentered bifocal, trifocal, linear or nonlinear aspheric multifocal and the like. These power profiles depend on the add power, the number of zones and their arrangement and location, the zone dimensions, and the depth of focus of the eye with near and distant focusing.

Depending on the layout of the optical zones of the cornea, one or more relative minima in the near and distant visual acuities will be present at various locations. For example, for circular optical zones, the distant acuity may have a minimum at the outer diameter of each zone corrected for near. Similarly, the near acuity may have a relative minimum at the outer diameter of each zone corrected for distance. The calculations of expected acuity are performed at these locations, to ensure that the minimum near and distant acuities meet the patient's requirements.

Using the pupil measurements, the aberration data, near and distant focusing, and the optical power profile for a series of optical zone dimensions and decentrations, a series of mathematical models of the wavefront of the eye/multifocal optic system is created. The spatial variation of the phase of the wavefront is determined by known mathematical formulas based on the optical power, shape and dimensions of each corrected zone of the cornea (Ref Munnerlyn), and the optical power and aberrations of the eye (Ref Liang). The amplitude of the light entering the eye is assumed to be uniform, and an abrupt transition to zero amplitude is assumed at the dimensions of the pupil. Alternatively, the amplitude may be nonuniform if opacities of the eye or correcting optics are known.

The modulation transfer function is calculated for each mathematical model of the wavefront, according to known formulas (Refs Born and Gaskill).

Preferably, the contrast threshold of the patient's right and left eyes is measured, at a number of spatial frequencies, for example about 15, 20, 24, 30, and 45 cycles per degree. Alternatively, the average values of the human retinal contrast threshold, as known in the scientific literature (Ref Green (1978)), are used. These thresholds are input to the computer, which calculates a contrast threshold function by linear interpolation of the measured values.

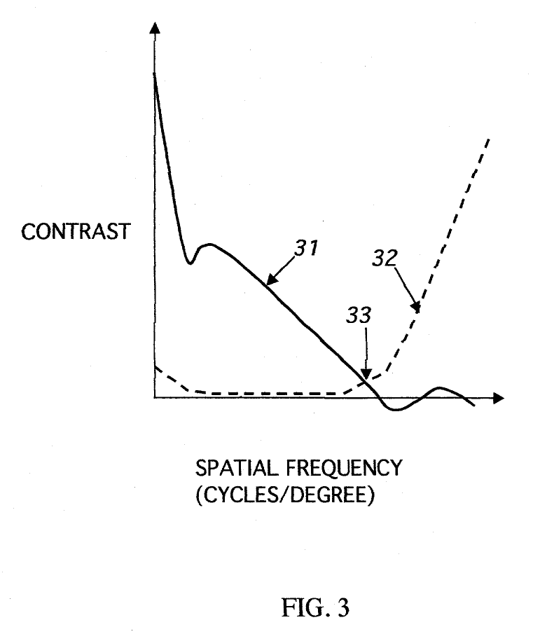

The maximum resolvable spatial frequency is the frequency at which the modulation transfer function reduces the contrast below the retinal contrast threshold. This is calculated for each transfer function. FIG. 3 schematically depicts an example modulation transfer function 31, contrast threshold function 32, and maximum resolvable spatial frequency 33.

The expected near and distant visual acuities are calculated as functions of the zone dimensions and decentration. The expected visual acuity is proportional to the maximum resolvable spatial frequency, with about 30 cycles per degree corresponding to 20/20 acuity. Tables or graphs of the expected near and distant visual acuity as functions of the zone dimensions and decentration are developed for each selected zone arrangement and power profile. The patient's near and distant visual acuity requirements are compared to the expected near and distant acuity. The zone dimensions and arrangements, decentrations and power profiles, which meet the patient's acuity requirements, are selected. Minimum and maximum acceptable values of the zone dimensions and decentration are determined, creating regions of zone size tolerance and a region of decentration tolerance. For certain cases, nomograms are developed, giving the acceptable range of zone dimensions as a function of the pupil dimensions.

Within the constraints of the minimum and maximum zone sizes and decentration, the design is optimized to meet patient requirements. Maximizing the size of the zones corrected for near maximizes the near acuity, while maintaining adequate distant acuity. Minimizing the size of the zones corrected for near maximizes the distant acuity and minimizes the side effects of glare and halos, while preserving the required near acuity. Selecting zone diameters in the middle of the acceptable ranges maximizes the tolerance for error in pupil measurement and error in the zone size and centration. Minimizing the decentration may minimize aberrations and improve the acuity.

Across the region of zone size tolerance, the optical power changes from predominantly distant correction to predominantly near correction. In this region, the optical power can vary abruptly with position, as in bifocal and trifocal optics, or smoothly, as in linear and nonlinear aspheric progressive optics. Smooth aspheric blends may reduce the diffractive effects of glare and halos, but they can induce aberrations, limiting the acuity. Therefore, aspheric blends are limited to be predominantly within the region of zone size tolerance, leaving zones predominantly corrected for near and distance. Within the zones corrected predominantly for near, the power profile differs from the nominal near correction power by less than the depth of focus of the eye, with near focusing. Similarly, within the zones corrected predominantly for distance, the power profile differs from the nominal distant correction power by less than the depth of focus of the eye, with distant focusing.

After the zone sizes, power profiles, and decentration are determined, a mathematical algorithm or table is created, defining the required postoperative multifocal profile of the corneal surface. This algorithm is transferred to the computer controlling a device, which reshapes the cornea or a corneal implant. Numerous devices used for corneal reshaping are known in the literature and patent art. Examples of such devices include lasers, thermal, mechanical, or electrical conductive devices, and others.

The method is illustrated by means of several examples. The examples share certain geometric features with the prior art (U.S. Pat. Nos. 5,533,997, 5,628,784, 5,803,923, 5,928,129, 6,059,775, 6,090,141, 6,280,435, 6,302,877). However, unlike the prior art, the invention corrects the entire optical zone of the cornea within the pupil, it corrects near and distant vision, and it optimizes the size and location of the zones, using the full set of required data. These improvements over the prior art are expected to improve the near and distant visual acuity, particularly for patients with small pupils, minimize potentially debilitating side effects of glare and halos, and reduce the number of patients who suffer loss of acuity after multifocal refractive surgery.

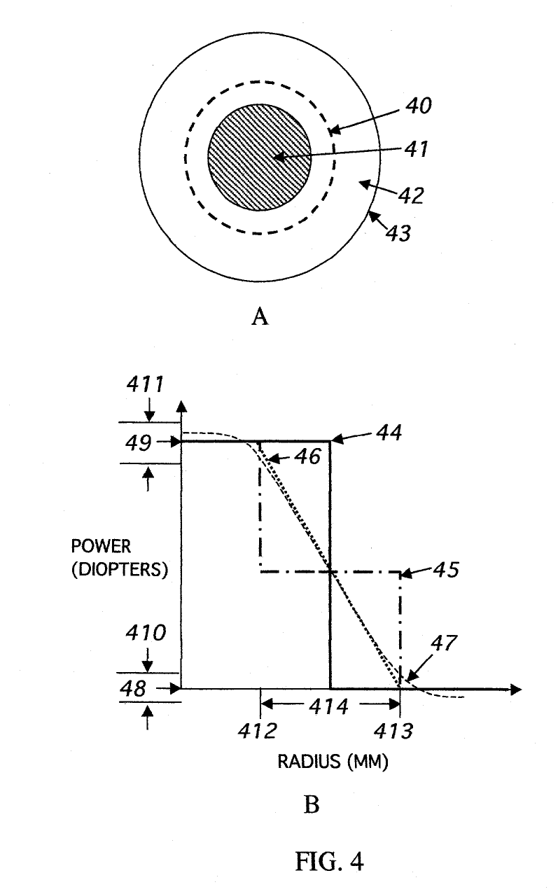

Consider two-zone, circular multifocal refractive surgery, with the central optical zone corrected predominantly for near. The arrangement of the zones is shown in FIG. 4A, with a pupil 40, a central zone 41 corrected for near, and a peripheral zone 42 corrected for distance. The outer diameter 43 of the peripheral zone is larger than the pupil dimension in dim light with distant focusing. Example optical power profiles are given in FIG. 4B, including bifocal 44, trifocal 45, linear aspheric multifocal 46, and smooth, nonlinear, aspheric, multifocal 47. The nominal powers corrected for distance 48 and near 49 are selected, and the depths of focus at distance 410 and near 411 are measured. The method of the patent is used to determine the minimum 412 and maximum 413 acceptable dimensions of the central optical zone, giving a region of zone tolerance 414.

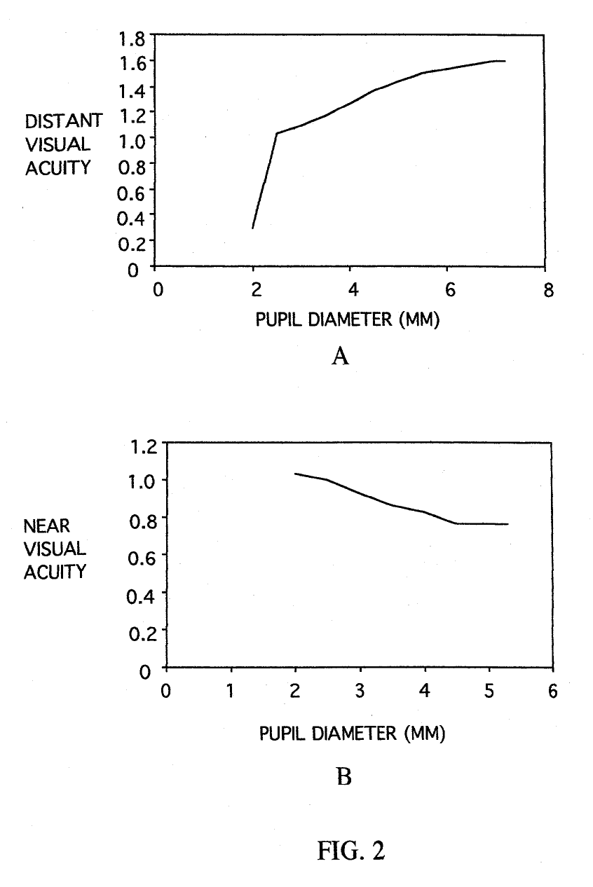

The zone dimensions are optimized, based on calculations of the expected near and distant acuity. As was noted in FIG. 2A, the expected distant visual acuity with this design is at a minimum for small pupils. Therefore, the expected distant acuity is calculated using measurements of the pupil dimensions in bright light with distant focus. Similarly, since the near visual acuity is at its minimum for large pupils, the near acuity calculations are based on the patient's pupil dimensions with near focusing in dim light. Preferably, the patient's retinal contrast threshold, ocular aberrations and depth of focus are measured and entered into a computer. A series of mathematical models of the phase and amplitude of the wavefront is developed, for a series of central optical zone dimensions, based on the data and known formulas. The modulation transfer function and the expected near and distant visual acuity are calculated, as functions of the central optical zone dimension. The dimensions, which provide adequate near and distant acuity, are selected. In FIG. 4B, the minimum 412 and maximum 413 radii define the region of zone tolerance 414.

Within the tolerance, an optimum central zone diameter is chosen. Maximizing the central zone diameter maximizes the near acuity, while minimizing the diameter maximizes the distant acuity and minimizes glare and halos. Selecting the average of the minimum and maximum diameters minimizes the effect of uncertainty or error in measurements or reshaping. Trifocal 45 and aspheric multifocal optics 46 and 47 are located predominantly in the region of zone tolerance 414. In the central zone, the aspheric multifocal power 47 differs from the nominal power for near correction 44 by less than the depth of focus of the eye, for near focusing 411. Similarly, in the peripheral zone, the aspheric power 47 differs from the nominal power for distance correction 48 by less than the depth of focus, for distant focusing 410.

The decentration is optimized in a similar fashion. A series of wavefronts and modulation transfer functions is calculated, for a series of decentrations. The expected visual acuity is calculated and compared to the required acuity. A range of acceptable values of the decentration is determined. Within the range, the decentration is optimized for the patient. For example, minimizing the decentration minimizes aberrations, and may improve the acuity. In this geometry, minimizing the decentration may also maximize the near acuity, while maximizing the decentration may maximize the distant acuity, in bright light. Selecting the decentration in the center of the acceptable range may minimize the effect of errors in measurement or reshaping.

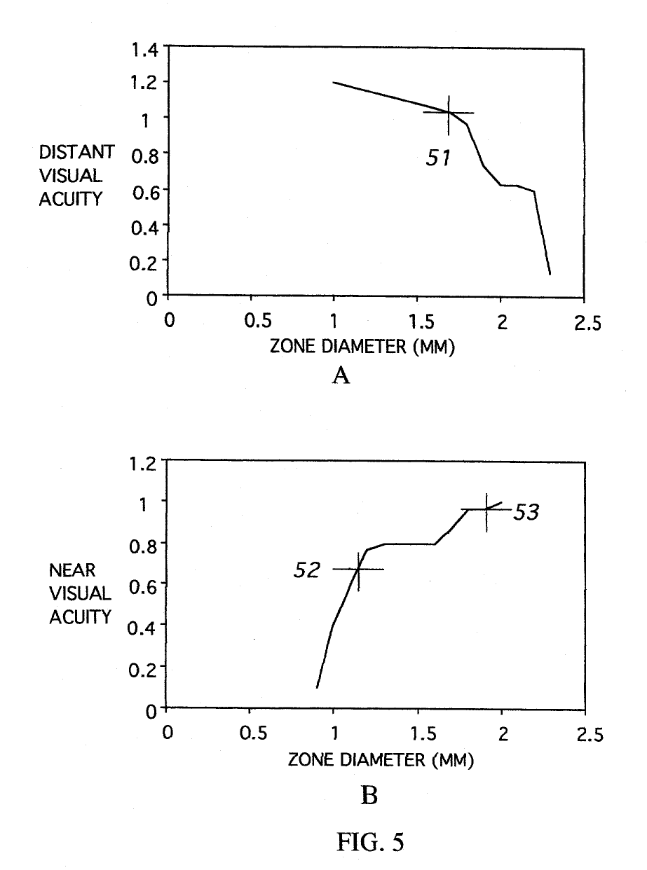

To illustrate the method, the optical zone dimensions are optimized for a centered, circular, 2-zone bifocal optic, with the central optical zone corrected for near. Take the case of a patient with insignificant aberrations, an average contrast threshold, required near acuity J2 (decimal 0.67), required distant acuity 20/20 (decimal 1.0), and average pupil dimensions, as reported in the literature. The pupil diameter in dim lighting with near focus is 3.5 mm, while the pupil diameter in bright lighting with distant focus is 2.6 mm. Using the method of this invention, a series of transfer functions is calculated, and the expected distant and near acuities are graphed versus the zone dimensions in FIG. 5.

Referring to FIG. 5A, the expected distant acuity exceeds 1.0 whenever the zone dimension is less than or equal to 1.7 mm, at the point labeled 51. Similarly, FIG. 5B shows that the expected near acuity exceeds 0.67 whenever the zone dimension is greater than or equal to 1.2 mm, at the point labeled 52. Therefore, the acceptable range or tolerance of zone dimensions for this patient is 1.2 to 1.7 mm. However, if the patient required J1+(decimal 1.0) near visual acuity, as in the point labeled 53, the minimum diameter of the central optical zone would be 2.0 mm, and the maximum diameter would remain 1.7 mm. Thus, there would be no optical zone dimension meeting the near and distant acuity requirements. The method disclosed in this patent optimizes the zone dimensions, and also identifies patients with unrealistic acuity expectations.

Nomograms are developed, giving the acceptable zone dimensions and tolerances as functions of the pupil measurements and acuity requirements, for centered, circular, two-zone multifocal optics, based on insignificant aberrations and normal contrast thresholds (see Tables 2 and 3). The maximum diameter of the central zone is further limited to be smaller than the pupil dimension in dim light with near focusing. The diameter of the peripheral zone is chosen to be larger than the pupil dimension in dim light with distant focusing.

Using this geometry, there is no single central optical zone size, meeting the near and distant acuity requirements across the population's entire range of pupil measurements. Therefore, it is necessary to select the central zone size based on measurements of the pupil dimensions for each patient. However, the design nomogram can be simplified by selecting a single zone dimension for all patients with pupils larger than a certain size. For example, all patients with pupil diameters greater than or equal to 2.5 mm can obtain J2 near acuity and 20/20 distant acuity with a 2-zone, center near multifocal ablation, if the central zone diameter is 1.7 mm.

By decentering the zones, it may be possible to select a single 2-zone, center-near circular arrangement, which meets the acuity requirements for all patients. This would eliminate the need for multiple measurements of pupil dimensions. The central zone dimension of a decentered multifocal corneal profile could exceed the pupil dimension in bright light, provided that the decentration is sufficient to give an adequate share of the aperture for both distant and near focusing. The minimum amount of decentration is that which provides adequate distant acuity in bright light, while the maximum decentration is that which provides adequate near acuity in bright light. The method of the patent is used to calculate the acceptable range of decentration, based on the zone dimensions and pupil dimensions. For this zone geometry, the decentration must be slightly more than half the central optical zone diameter. For example, the minimum size of the central zone which meets a J2 near acuity requirement in dim light for all patients is about 1.7 mm. Decentering this zone about 1.0 to 1.1 mm may provide adequate distant and near acuity.

In order to provide adequate distant acuity as the pupil becomes smaller with age, the maximum central zone size is reduced, while maintaining the minimum value. For example, consider the case of the patient with an average pupil dimension with distant focusing in bright light of 2.6 mm, and a distant acuity requirement of 20/20 (decimal 1.0), desiring continued correction of presbyopia for 10 years. In 10 years, the patient's pupil is expected to decline from 2.6 to 2.3 mm (Ref Koch). Using the nomogram in Table 2, reducing the pupil dimension from 2.6 mm to 2.3 mm reduces the maximum diameter of the central optical zone from 1.7 to 1.5 mm. In this example, such a reduction is acceptable, because the minimum diameter of the central optical zone is 1.2 mm.

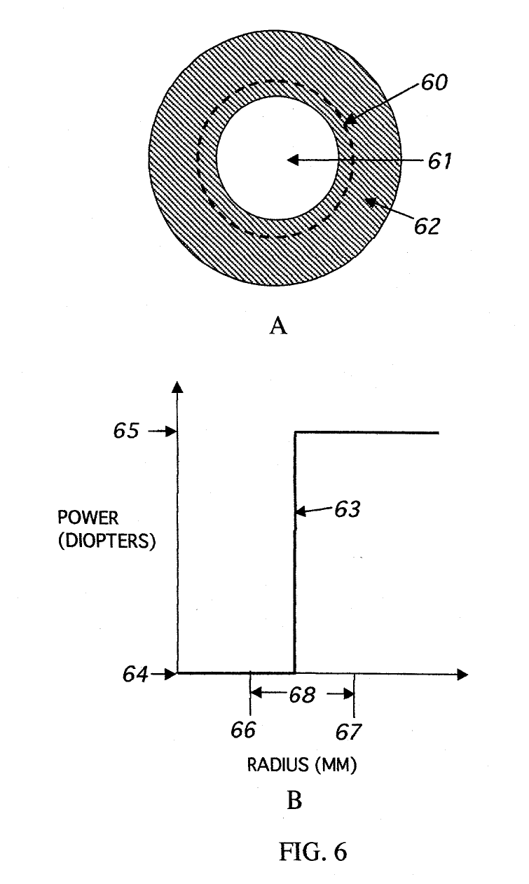

Now consider the case of two circular zones, with the center zone corrected for distance. FIG. 6A shows the layout of the zones, with a pupil 60, a central zone 61 corrected for distance, and a peripheral zone 62 corrected for near. FIG. 6B shows an example bifocal power profile 63, with a nominal power corrected for distance 64, and a nominal power corrected for near 65. Trifocal and aspheric profiles have constraints as discussed in the center-near geometry, but are omitted from the figure for clarity. The method of the patent is used to calculate the minimum 66 and maximum 67 central zone radii, giving a region of zone tolerance 68. The distant acuity is at its minimum for large pupils, while the near acuity is at its minimum for the larger of the central optical zone diameter or the pupil dimension in bright light with near focusing. Modulation transfer functions and expected near and distant visual acuities are calculated as functions of the central zone diameter and decentration. The acceptable range of zone dimensions and decentrations is determined, and the patient's optimum zone diameter and decentration are selected.

Nomograms are developed for centered, circular zones, average retinal contrast threshold, and insignificant aberrations (Tables 4 and 5). Consider the example of a patient with average pupil dimensions, a required distant acuity of 20/20, and a required near acuity of J2. The pupil diameter in dim light with distant focus is 5.3 mm, and Table 4 shows that the minimum central zone diameter is 2.7 mm. The pupil diameter in bright light with near focus is 3.0 mm, and Table 5 gives a maximum central zone diameter of 2.6 mm. Thus, there is no optical zone dimension at which the acuity requirements of this average patient are met, using this geometry. The calculations show that the two-zone, center-distant geometry meets these acuity requirements only for patients with the largest pupils. Because the peripheral optical zone has such a large fraction of the area of the pupil, nighttime glare and halos are predicted to be more severe for this zone arrangement than for the two-zone, center-near arrangement.

Decentering the zones would allow the central zone to be larger, potentially providing adequate near and distant acuity for a larger number of patients. The decentration is selected to provide an adequate share of the pupil aperture corrected for near and distant focusing. The method of the patent is used to calculate the acceptable range of decentration, based on the zone dimensions and pupil dimensions. For this geometry, the decentration must be slightly less than half the central zone diameter. For example, the minimum size of the central zone which meets a 20/20 distant acuity requirement in dim light for all patients is about 3.4 mm. Decentering this zone about 1.45 to 1.55 mm may provide adequate distant and near acuity. Within the range of acceptable decentrations, the design is optimized as in the two-zone, center-near case.

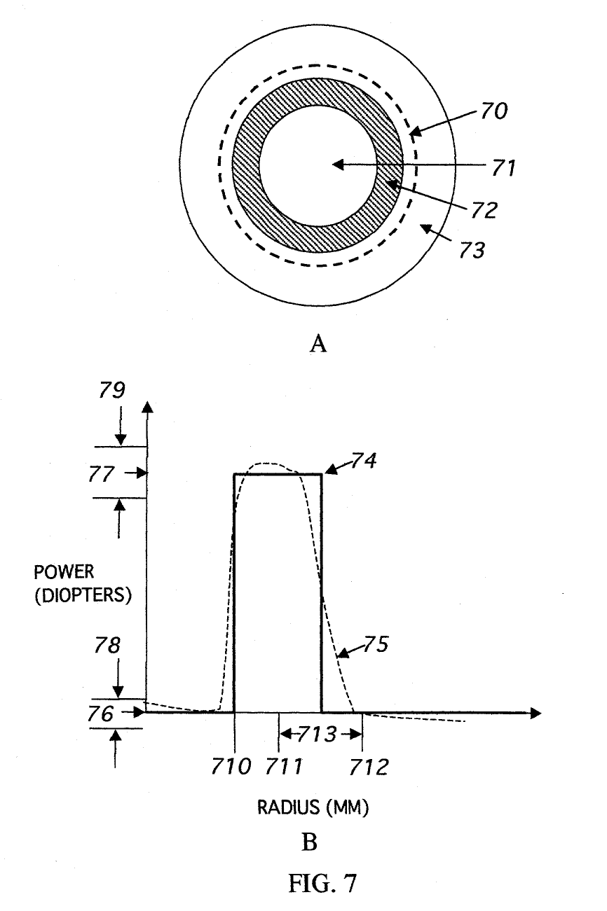

Now consider the case of three circular optical zones. The layout of three zones is shown in FIG. 7A, with a pupil 70, a central zone 71 and a peripheral zone 73 corrected for distance, and a midperipheral zone 72 corrected for near. Selecting this layout, rather than correcting the peripheral zone for near, minimizes glare and halos. The minimum distant acuity occurs when the pupil dimension equals the larger of the diameter of the midperipheral zone, or the pupil dimension in bright light with distant focusing. Relative minimums in the near acuity occur for large and small pupils. The peripheral optical zone diameter is larger than the pupil dimension in dim light with distant focus. FIG. 7B shows an example bifocal power profile 74, and an aspheric multifocal profile 75. The nominal powers at distance 76 and near 77 are determined, and the depth of focus at distance 78 and near 79 are measured. The method of the patent determines the maximum central zone radius 710, and the minimum 711 and maximum 712 midperipheral zone radii, yielding the region of zone tolerance 713.

There is, in general, no minimum diameter of the central optical zone. In the case of a central optical zone with a diameter of zero millimeters, the geometry is identical to the two-zone, center-near arrangement. In order to provide the maximum visual acuity at distance under bright lighting, the central optical zone diameter is maximized, while maintaining adequate near acuity with bright lighting. The minimum outer diameter of the midperipheral zone is determined by the requirement to attain adequate near acuity with dim lighting. The maximum outer diameter of the midperipheral zone is determined by the requirement to attain adequate distant acuity. The midperipheral zone diameter is further limited to be less than the pupil dimension with dim lighting and near focusing.

Wavefront models, transfer functions, and the expected near and distant acuities are calculated as functions of the zone dimensions and decentration. The midperipheral zone dimensions and decentrations, which meet the near and distant acuity requirements, are selected. Trifocal and aspheric zones are placed predominantly within the region of zone tolerance, subject to the constraints noted above in the two-zone, center-near case. Within the constraints of the minimum and maximum midperipheral zone sizes, the design is optimized as in the case of two zones to meet patient requirements.

Nomograms are developed for the case of three centered, circular zones, insignificant aberrations, average retinal contrast sensitivity, a required distant acuity of 20/20, and a required near acuity of J2. Table 6 gives the maximum diameter of the central zone and the corresponding maximum dimensions of the midperipheral zone, as a function of the pupil dimension under bright lighting and near focusing. Table 8 gives the minimum diameter of the midperipheral zone as a function of the pupil dimension with dim light and near focusing, and the central zone diameter.

For very small pupils, the minimum diameter of the central zone is less than 1.8 mm. In these cases, an iterative process is necessary to determine the zone dimensions. First, the maximum central zone diameter is determined from Table 6. Next, the maximum midperipheral zone diameter is derived from Table 7. Then the minimum midperipheral zone diameter is derived from Table 8. If the minimum is less than the maximum, the values are selected. However, if the minimum exceeds the maximum, the initial choice of the central zone diameter was too large, and it is reduced by 0.1 mm. Tables 7 and 8 are used again to determine the minimum and maximum midperipheral zone dimensions. The process is iterated until the minimum diameter is less than the maximum diameter.

As in the case of two zones, the central zone diameter is modified to continue to correct presbyopia as the pupil diameter declines with age. For example, consider the case of the patient with an average pupil dimension of 3.0 mm with near focusing in bright light, and a near acuity requirement of J2 (decimal 0.67). In 10 years, the patient's pupil is expected to decline to 2.7 mm. Using the nomogram in table 6, the maximum diameter of the central optical zone is reduced from 2.6 to 2.2 mm, and the midperipheral optical zone minimum diameter is reduced from 3.3 to 2.9 mm.

For the centered, circular, three-zone geometry, there is no single set of zone sizes meeting the near and distant acuity requirements for all patients. Therefore, it is necessary to select the central zone size based on pupil measurements of individual patients. However, the nomogram can be simplified, for example, by selecting all patients with pupils measuring 2.5 mm or greater. For these patients, a distant acuity of 20/20 and a near acuity of J2 are expected, if the add zone has an inner diameter of 1.8 mm and an outer diameter of 2.8 mm.

Decentering the zones may allow use of a single set of zone sizes for all patients, using this geometry. Modulation transfer functions and expected acuities are calculated versus the decentration, and the acceptable range of decentrations is determined. Decentering the multifocal profile, by an amount about 0.2 to 0.25 mm less than half the diameter of the central optical zone, may provide adequate near and distant acuity, even for patients with the smallest pupil dimensions known in the literature. For example, decentering an add zone, with an inner diameter of 1.8 mm and an outer diameter of 2.8 mm, by 0.65-0.7 mm may provide adequate distant and near acuity for a large number of patients.

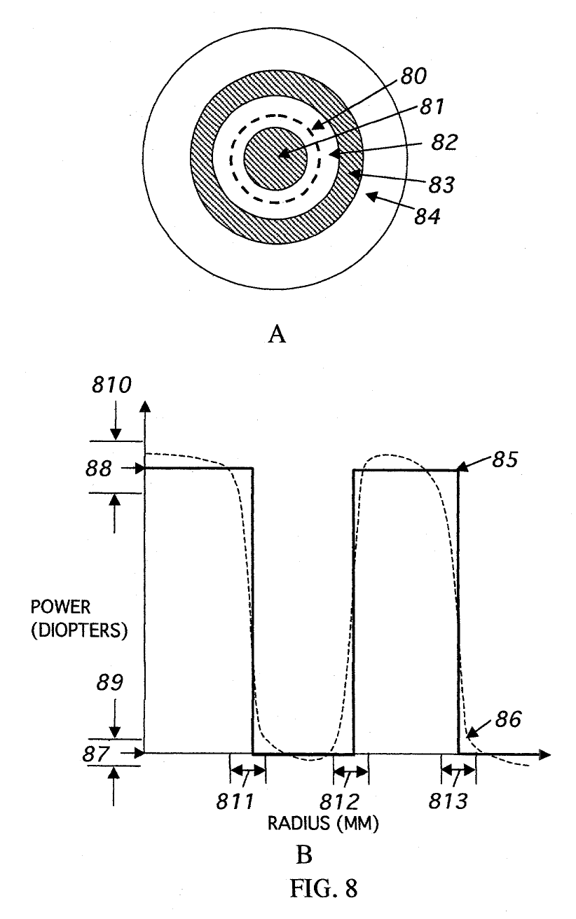

Four-zone circular multifocal refractive surgery is depicted schematically in FIG. 8. The pupil 80, the zones 81 and 83 corrected for near, and the zones 82 and 84 corrected for distance are shown in FIG. 8A. Compared to correcting the peripheral zone for near, geometry minimizes glare and halos. FIG. 8B shows an example bifocal power profile 85 and an aspheric profile 86, with nominal powers corrected for distance 87 and near 88. The depth of focus at distance 89 and near 810 is measured. The method of the patent is used to determine the regions of zone tolerance 811, 812, and 813. The outer diameter of the peripheral zone is greater than the pupil dimension in dim light with distant focusing.

Relative minima in the near visual acuity occur when the pupil dimension equals the outer diameter of the second optical zone 82 and at the pupil dimension with dim light and near focusing. Relative minima in the distant acuity occur at the outer diameters of the central optical zone 81 and the third optical zone 83. The near and distant acuities are calculated at their relative minima. The range of pupil dimensions of the entire population (Ref Koch) are used in the calculations. A series of transfer functions, and expected near and distant acuities are calculated as functions of the zone dimensions and decentrations. The zone dimensions and decentrations meeting the acuity goals are determined, defining the regions of tolerance. Within the tolerances, the zone dimensions, decentration, and aspheric blends are optimized for the patient's needs, as in the 2-zone, center-near case. This allows programming of the device which reshapes the cornea or the corneal implant.

Assuming centered, circular zones, insignificant aberrations, average contrast threshold, 20/20 required distant vision and J2 required near vision, the nomogram in Table 9 gives the acceptable zone dimensions and their tolerances.

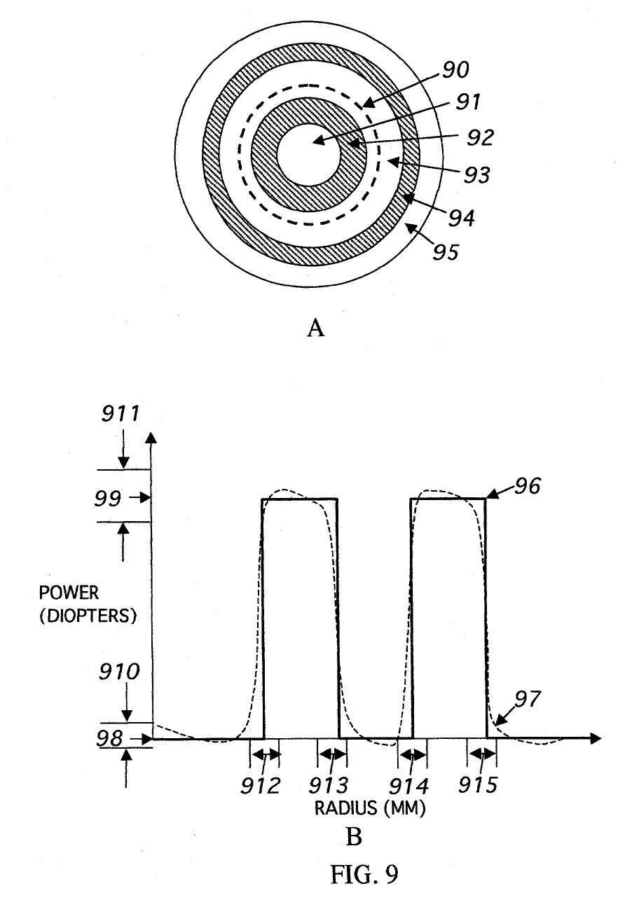

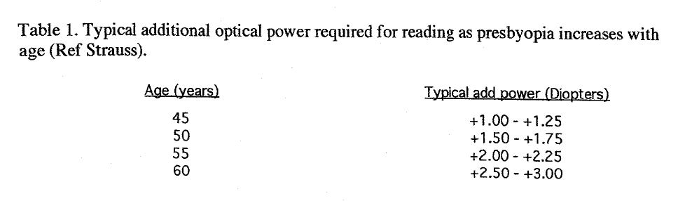

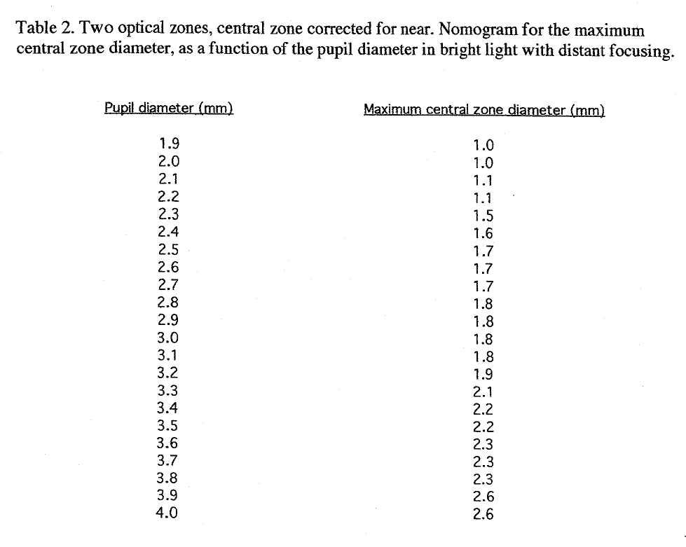

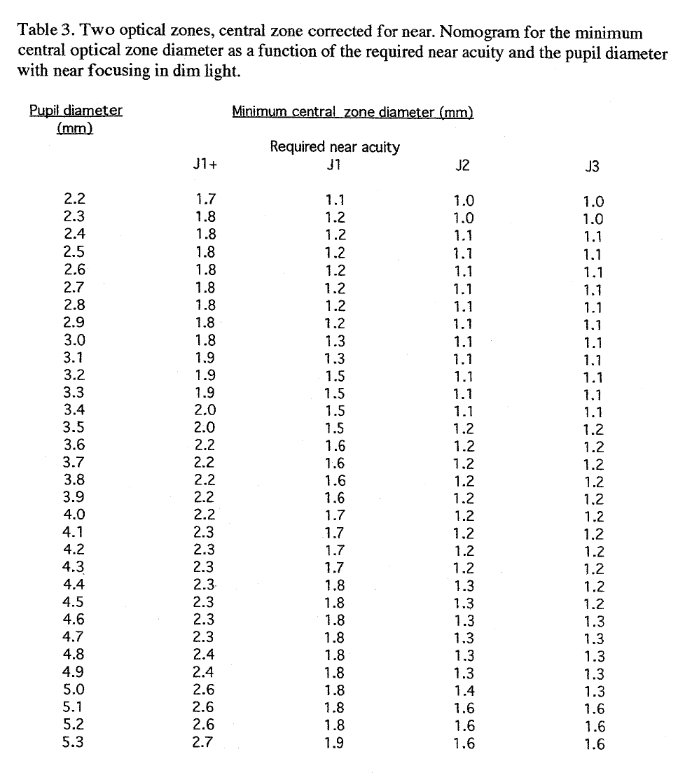

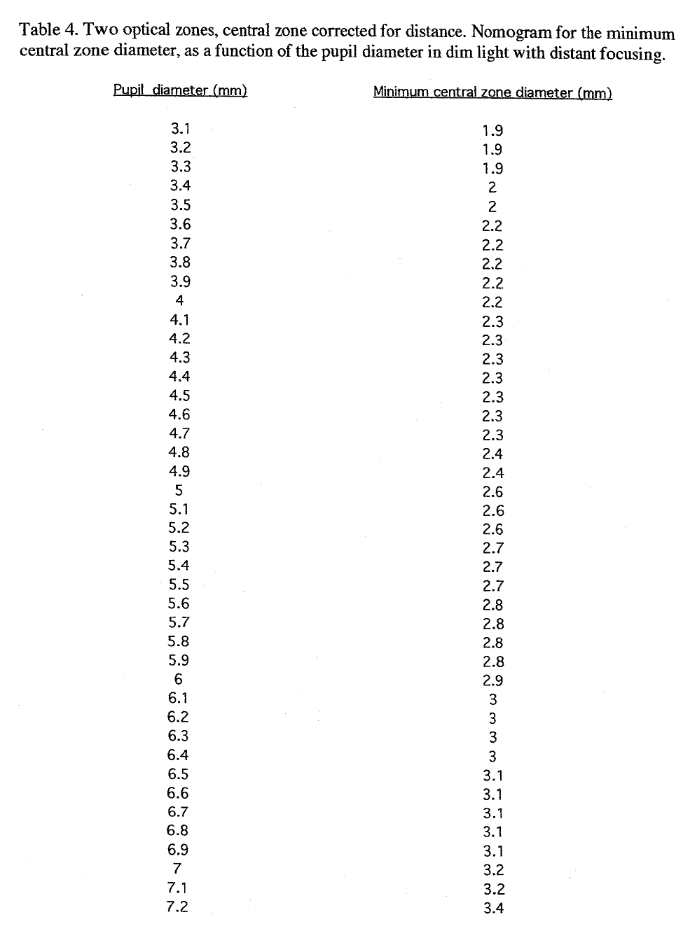

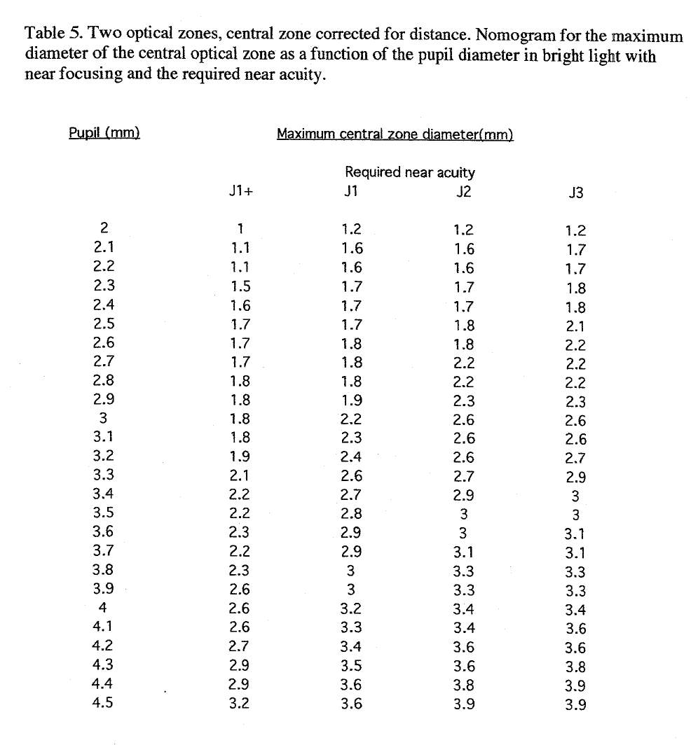

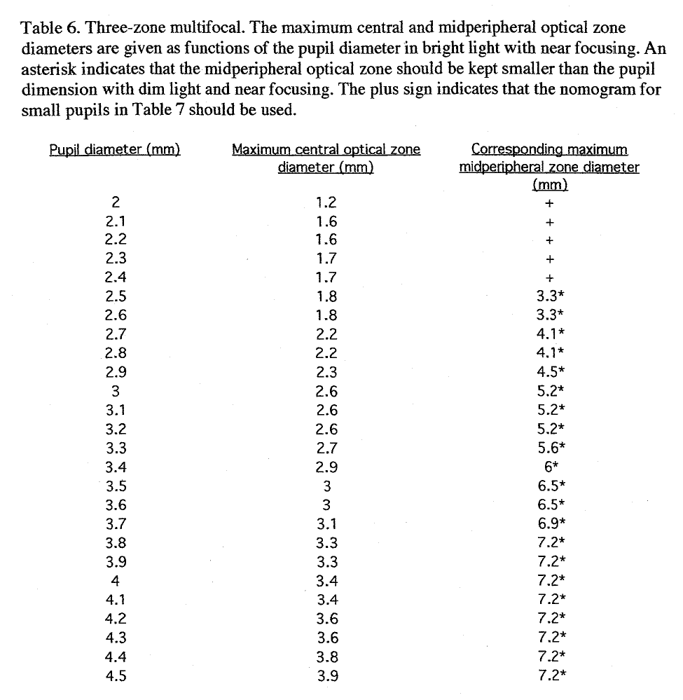

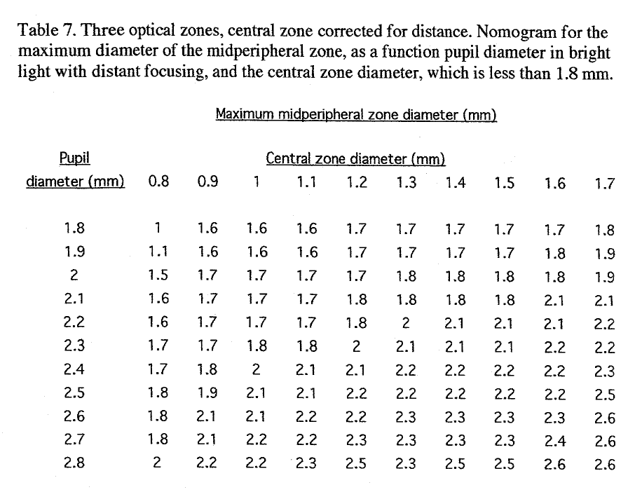

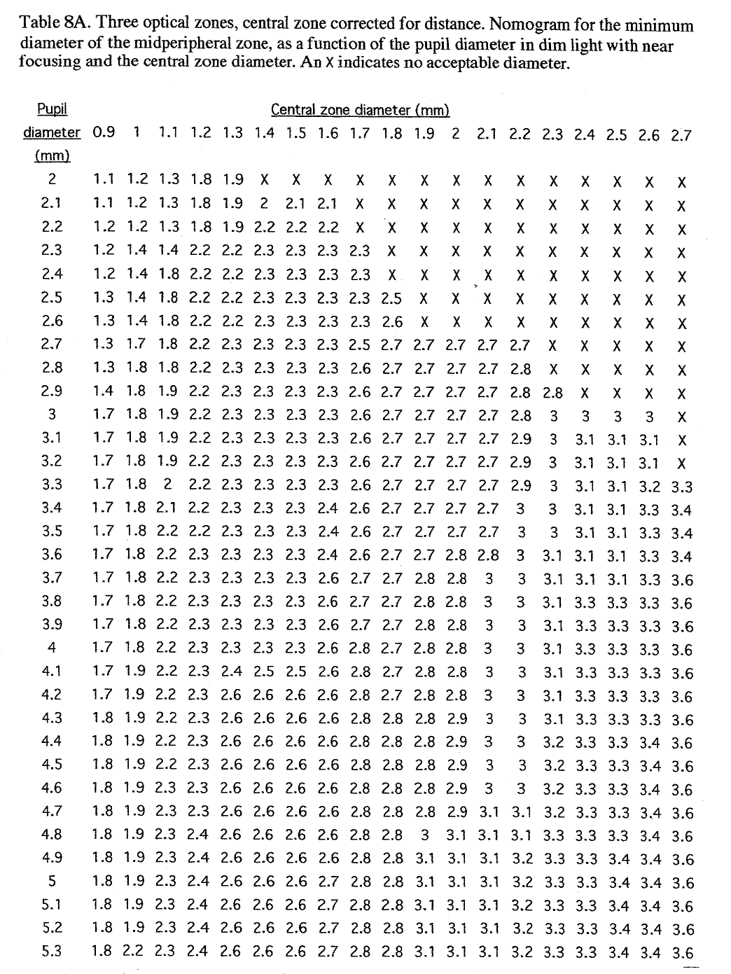

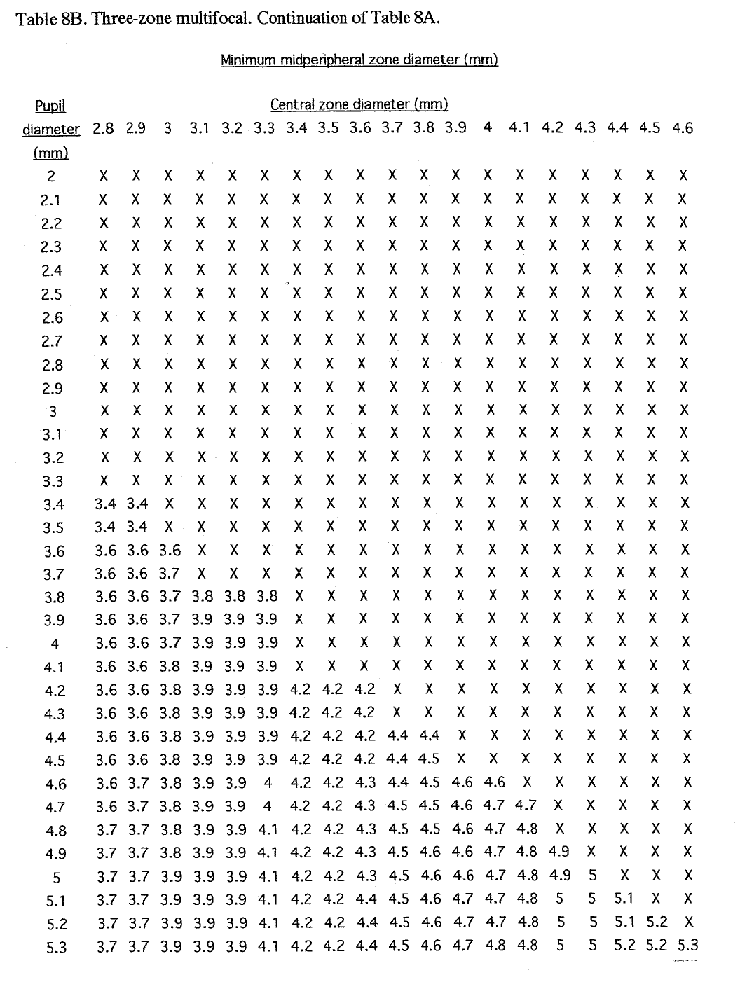

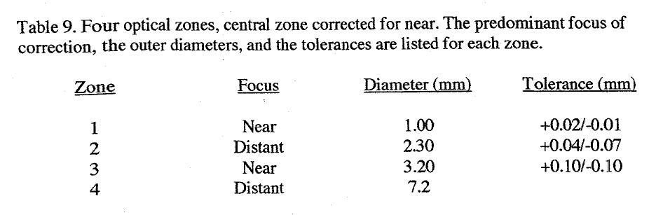

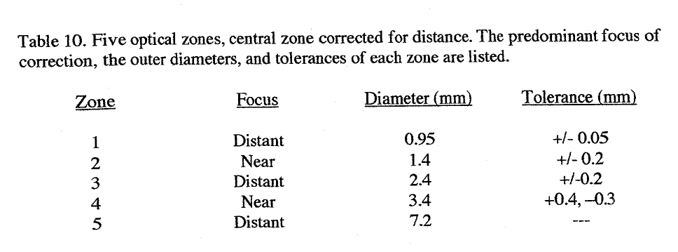

Multifocal refractive surgery with five circular zones is shown schematically in FIG. 9A. The pupil 90, the zones 91, 93, and 95 corrected for distance, and the zones 92 and 94 corrected for near are indicated. Compared to correcting he peripheral zone for near, this geometry minimizes glare and halos. In FIG. 9B, bifocal 96 and aspheric multifocal 97 power profiles are shown, with nominal powers corrected for distance 98 and near 99. The depth of focus at distance 910 and near 911 is measured. The method of the patent determines the allowable decentration and the regions of zone tolerance 912, 913, 914 and 915. The diameter of the peripheral zone 95 is greater than the pupil dimension in dim light with distant focusing.

Relative minima in the near acuity occur when the pupil dimension equals the outer diameter of the central and third zones, and in dim light with near focusing. Relative minima in the distant acuity occur at the outer diameters of the second and fourth zones. The near and distant acuity calculations are performed at their relative minima, across the entire range of pupil dimensions of the population (Ref Koch). The regions of zone tolerance and allowable decentration are calculated. The dimensions, decentration and aspheric blends are selected, and the programmed device reshapes the cornea or implant.

Assuming centered, circular zones, normal contrast sensitivity, insignificant aberrations, and near and distant acuity requirements of J2 and 20/20 respectively, the nomogram in Table 10 gives the acceptable zone dimensions and their tolerances.

Multifocal corneal refractive surgery can be performed with six, seven, or more optical zones. In order to minimize glare and halos, the peripheral optical zone should be corrected predominantly for distance. Regardless of the number of zones, they may be circular or noncircular, centered or decentered, and may have abrupt steps in the power or smooth aspheric blends. The method of this patent is used to select the dimensions and decentration of the optical zones. The device reshaping the cornea or implant is programmed, based on the optimized zone dimensions and decentration.

These descriptions are given by way of example, and numerous other examples will be apparent to those skilled in the art, within the scope of the patent. The invention is limited solely as stated in the claims.

I claim:

1. A method for performing multifocal refractive surgery on the cornea of an eye, said method correcting presbyopia, myopia, hyperopia, and regular and irregular astigmatism, said method creating a plurality of optical zones, at least one of said zones being corrected predominantly for near vision and at least one of said zones being corrected predominantly for distant vision, said method optimizing the dimensions of said optical zones, said method comprising:
(i) preferably, measuring the pupil diameters of patient's eyes in bright and dim light with near and distant focusing;
(ii) alternatively, using the range of pupil measurements of the entire population, in bright and dim light, with near and distant focusing, said range being known in the literature;
(iii) determining the patient's near and distant visual acuity requirements;
(iv) measuring the refractive error of the patient's eyes;
(v) preferably, measuring the optical aberrations of the patient's eyes;
(vi) alternatively, assuming that the patient's optical aberrations are insignificant;
(vii) preferably, measuring the patient's retinal contrast threshold as a function of the spatial frequency;
(viii) alternatively, using the average human retinal contrast threshold as a function of the spatial frequency, which is known in the literature;
(ix) selecting the overall dimension of the outermost corrected optical zone to exceed the pupil dimension in dim lighting with distant focusing;
(x) selecting for investigation a candidate arrangement of the optical zones, such as correcting the central zone for distance, and other zones for near and intermediate focusing;
(xi) selecting for investigation a candidate optical zone geometry, such as circular, annular, sectoral, ovoid and the like;
(xii) selecting for investigation a candidate nominal optical power for each zone, to predominantly correct near, distant or intermediate visual acuity;
(xiii) selecting for investigation a candidate decentration of the optical zones from the center of the pupil;
(xiv) selecting for investigation a candidate optical power profile as a function of the location, such as bifocal, trifocal, linear or nonlinear aspheric multifocal and the like;
(xv) using the pupil measurements, focusing, optical power profile, and aberrations, together with a series of optical zone dimensions, to create a series of mathematical models of the wavefront transmitted through the eye/multifocal optic system;
(xvi) calculating the modulation transfer function for each said mathematical model of the wavefront;
(xvii) calculating the maximum resolvable spatial frequency for each said modulation transfer function;
(xviii) calculating the expected visual acuity for each said modulation transfer function;
(xix) creating tables or graphs of the expected near and distant visual acuity as functions of the zone dimensions;
(xx) comparing the patient's required near and distant visual acuity to the expected visual acuity as a function of the zone dimensions;
(xxi) selecting the zone dimensions which provide expected visual acuity greater than or equal to the visual acuity requirements;
(xxii) further limiting the dimensions of all optical zones corrected for near to be less than the pupil dimension in dim lighting with near focusing;
(xxiii) thereby determining the minimum and maximum zone dimensions;
(xxiv) selecting an optimum zone dimension from the ranges defined by said minimum and maximum zone dimensions;
(xxv) creating an algorithm or table defining the required postoperative profile of the surface of the cornea; and
(xxvi) thereby programming a computer-controlled laser, thermal, mechanical, electrical, or other device to reshape the cornea or a corneal implant, to produce said multifocal corneal surface profile.

2. The method of claim 1, wherein the near visual acuity is maximized, by maximizing the size of the zones corrected predominantly for near vision.

3. The method of claim 1, wherein the distant visual acuity is maximized, by minimizing the size of the zones corrected predominantly for near vision.

4. The method of claim 1, wherein the side effects of glare and halos are minimized, by minimizing the size of the zones corrected predominantly for near vision.

5. The method of claim 1, wherein the effects of inaccuracies in measurements of the pupil dimensions, inaccuracies in calculations of the zone dimensions or decentration, and inaccuracies in producing the zones are minimized, by selecting the mean of the minimum and maximum of the optical zone dimensions.

6. The method of claim 1, wherein the zones are circles and annuli, predominantly centered over the pupil, and wherein the required distant acuity is 20/20, and wherein the required near acuity ranges from J3 to J1+, and wherein nomograms provide the zone dimensions, tolerances and arrangement as functions of the pupil measurements.

7. The method of claim 6, wherein 2 optical zones are present, wherein the central zone is predominantly corrected for near and the peripheral zone is predominantly corrected for distance, using the nomograms in Tables 2 and 3.

8. The method of claim 6, wherein 2 optical zones are present, wherein the central zone is predominantly corrected for distance and the peripheral zone is predominantly corrected for near, using the nomograms in Tables 4 and 5.

9. The method of claim 6, wherein 3 optical zones are present, wherein the central and peripheral zones are predominantly corrected for distance and the midperipheral zone is predominantly corrected for near, using the nomograms in Tables 6, 7, and 8.

10. The method of claim 6, wherein 4 optical zones are present, wherein the central and third zones are predominantly corrected for near and the second and peripheral zones are predominantly corrected for distance, using the nomogram in Table 9.

11. The method of claim 6, wherein 5 optical zones are present, wherein the central, third, and peripheral zones are predominantly corrected for distance, and the second and fourth zones are predominantly corrected for near, using the nomogram in Table 10.

12. The method of claim 6, wherein the nomogram consists of a single set of zone dimensions for all patients having pupil diameters greater than a certain value.

13. The method of claim 1, wherein one or more trifocal zones, having about half the additional optical power required for near vision, reside between the distant and near corrected zones, predominantly within the region of zone tolerance, which is the region bounded by the minimum and maximum zone dimensions.

14. The method of claim 1, wherein one or more aspheric blend zones reside between the zones corrected primarily for near and distance vision, said aspheric blend zones being situated predominantly within the regions of tolerance of the optical zone dimensions, and said aspheric blend zones having an optical power profile within each zone corrected predominantly for near vision, said optical power differing from the nominal power of said near-corrected zone by less than a depth of focus of the eye with near focusing, and said aspheric blend zones having an optical power profile within each zone corrected predominantly for distant vision, said optical power differing from the nominal power of said distant-corrected zone by less than a depth of focus of the eye with distant focusing.

15. The method of claim 14, wherein the optical power within the aspheric blend zone is a linear function of the radius.

16. The method of claim 14, wherein the aspheric blend is a smooth, nonlinear function of the radius.

17. The method of claim 14, wherein the depth of focus of the eye is measured with near and distant focusing, for each individual patient.

18. The method of claim 14, wherein the depth of focus is the average depth of focus of the human eye, said average depth of focus being known in the literature.

19. A method as in claim 1 of performing multifocal refractive surgery on the cornea of an eye, said method adjusting the zone dimensions to ensure that the acuity requirements are met as the pupil dimension declines with age, said method consisting of:
(i) measuring the pupil dimensions in bright and dim light with near and distant focusing,
(ii) reducing the pupil dimension by about 0.3 mm per decade of additional age desired for correction,
(iii) recalculating the modulation transfer function, maximum frequency, and expected visual acuity as functions of the zone dimensions, arrangement and decentration, as in claim 1 or claim 19, based on the reduced pupil dimensions,
(iv) determining the patient's near and distant visual acuity requirements, and
(v) selecting the zone sizes, tolerances, decentration and arrangement, which meet the acuity requirements.

20. A method as in claim 1, step xii, of selecting a nominal additional optical power to predominantly correct near vision, said method correcting presbyopia, as presbyopia progresses with age, said method comprising the steps of:
(i) determining the patient's current additional optical power requirement for near vision,
(ii) measuring the patient's maximum tolerated additional optical power, using trial frame spectacles,
(iii) selecting a nominal additional optical power, equal to the patient's current additional optical power requirement, plus a supplementary optical power of about 1.0 Diopters per decade of additional age desired for correction, up to a maximum of about 2.75 to 3.0 Diopters, or the patient's maximum tolerated additional power, whichever is less.

21. A method for performing multifocal refractive surgery on the cornea of an eye, said method correcting presbyopia, myopia, hyperopia, and regular and irregular astigmatism, said method creating a plurality of optical zones, at least one of said zones being corrected predominantly for near vision and at least one of said zones being corrected predominantly for distant vision, said method optimizing the decentration of said optical zones, said method comprising:
(i) measuring the pupil diameters of patient's eyes in bright and dim light with near and distant focusing;
(ii) alternatively, using the range of pupil measurements of the entire population, in bright and dim light, with near and distant focusing, said range being known in the literature;
(iii) determining the patient's near and distant visual acuity requirements;
(iv) measuring the refractive error of the patient's eyes;
(v) preferably, measuring the optical aberrations of the patient's eyes;
(vi) alternatively, assuming that the patient's optical aberrations are insignificant;
(vii) preferably, measuring the patient's retinal contrast threshold as a function of the spatial frequency;
(viii) alternatively, using the average human retinal contrast threshold as a function of the spatial frequency;
(ix) selecting the overall dimension of the outermost corrected optical zone to exceed the pupil dimension in dim lighting with distant focusing;
(x) selecting for investigation a candidate optical zone geometry, such as circular, annular, sectoral, ovoid and the like;
(xi) selecting for investigation a candidate arrangement of the optical zones, such as correcting the central zone for distance, and other zones for near and intermediate focusing;
(xii) selecting for investigation a candidate nominal optical power for each zone, to predominantly correct near, distant or intermediate visual acuity;
(xiii) selecting for investigation a candidate optical power profile as a function of the location, such as bifocal, trifocal, linear or nonlinear aspheric multifocal and the like;
(xiv) selecting for investigation a dimension for each of the optical zones;
(xv) using the pupil measurements, focusing, zone dimensions, optical power profile, and aberrations, together with a series of optical zone decentrations, to create a series of mathematical models of the wavefront transmitted through the eye/multifocal optic system;
(xvi) calculating the modulation transfer function for each said mathematical model of the wavefront;
(xvii) calculating the maximum resolvable spatial frequency for each said modulation transfer function;
(xviii) calculating the expected visual acuity for each said modulation transfer function;
(xix) creating tables or graphs of the expected near and distant visual acuity as functions of the zone decentrations;
(xx) comparing the patient's required near and distant visual acuity to the expected visual acuity as a function of the zone decentrations;
(xxi) selecting the zone decentrations which provide expected visual acuity greater than or equal to the visual acuity requirements;
(xxii) thereby determining the minimum and maximum zone decentrations;
(xxiii) selecting an optimum zone decentration from the ranges defined by said minimum and maximum zone decentrations;
(xxiv) creating an algorithm or table defining the required postoperative profile of the surface of the cornea; and
(xxv) thereby programming a computer-controlled laser, thermal, electrical, mechanical, or other device, to reshape the cornea or a corneal implant, to produce said multifocal corneal surface profile.
